(19) United States
(12) Patent Application Publication
(10) Pub. No.: US 2010/0081946 A1
(43) Pub. Date: Apr. 1, 2010

(54) METHOD AND APPARATUS FOR NON-INVASIVE CUFF-LESS BLOOD PRESSURE ESTIMATION USING PULSE ARRIVAL TIME AND HEART RATE WITH ADAPTIVE CALIBRATION

(75) Inventors: Harinath Garudadri, San Diego, CA (US); Federico S. Cattivelli, Los Angeles, CA (US); Pawan K. Baheti, San Diego, CA (US)

Correspondence Address: QUALCOMM INCORPORATED, 5775 MOREHOUSE DR., SAN DIEGO, CA, 92121, US

(73) Assignee: QUALCOMM Incorporated, San Diego, CA (US)

(21) Appl. No.: 12/547,982

(22) Filed: Aug. 26, 2009

Related U.S. Application Data
(60) Provisional application No. 61/100,626, filed on Sep. 26, 2008.
(60) Provisional application No. 61/101,078, filed on Sep. 29, 2008.

(51) Int. Cl. A61B 5/021

(52) U.S. Cl. 600/485

(57) ABSTRACT

Certain aspects of the present disclosure relate to a method for estimating a blood pressure using both a pulse arrival time (PAT) and an instantaneous heart rate (HR). The PAT can be measured as the delay between QRS peaks in an electrocardiogram (ECG) signal and corresponding points in a photoplethysmogram (PPG) waveform. Parameters of the estimation model can be determined through an initial training. Then, the model parameters can be recalibrated in constant intervals using the recursive least square (RLS) approach combined with a smooth bias fixing. The proposed estimation algorithm is applied on a multi-parameter intelligent monitoring for intensive care (MIMIC) database, and the results are compared with estimation methods that use PAT only or HR only. The proposed estimation algorithm meets, on average, the Association for the Advancement of Medical Instrumentation (AAMI) requirements and outperforms other methods from the prior art. It is also shown in the present disclosure that the proposed estimation algorithm is robust to unknown skew between the ECG and PPG signals.

### CLAIM OF PRIORITY UNDER 35 U.S.C. §119

The present Application for Patent claims benefit of Provisional Application Ser. No. 61/100,626 filed Sep. 26, 2008, and Provisional Application Ser. No. 61/101,078 filed Sep. 29, 2008 and assigned to the assignee hereof and hereby expressly incorporated by reference herein.

### BACKGROUND

1. Field

Certain aspects of the present disclosure generally relate to signal processing and, more particularly, to a method for non-invasive cuff-less blood pressure estimation based on pulse arrival time and heart rate.

2. Background

When measuring an arterial blood pressure (BP), two values are of particular interest—systolic blood pressure (SBP) and diastolic blood pressure (DBP). The SBP represents the peak pressure in arteries occurring near the beginning of every cardiac cycle, while the DBP represents the lowest pressure occurring at the resting stage of the cardiac cycle. The typical method for measuring the BP is known in the art as the ausculatory method, where a physician inflates a cuff around the patient's arm. Then, a stethoscope can detect pressures at which the brachial artery is occluded (for measuring the SBP) and released (for measuring the DBP). However, this method requires an expert (e.g., the physician) to perform the measurements.

Oscillometric technique for measuring the BP is based on the same principle but using a calibrated machine that automatically inflates the cuff and detects the vibrations. This particular method can be done safely at home, and does not require an expert to perform the measurements. However, both the ausculatory method and the oscillometric method require the use of a cuff, which is bulky, costly and usually requires a large power for automatic operation. Moreover, these methods do not allow continuous monitoring of blood pressure.

Another method to measure blood pressure is known in the art as the invasive method, where a catheter with a pressure sensor can be placed directly inside the artery. This method can provide the best accuracy and can give continuous monitoring of blood pressure. However, this method is invasive, which is the main disadvantage.

It has been shown in the prior art that the Pulse Transit Time (PTT) can be used to estimate SBP and DBP values. The PTT can be typically measured indirectly through a related quantity known as a Pulse Arrival Time (PAT). The PAT can be measured as the delay between QRS peaks in an electrocardiogram (ECG) signal and corresponding points in a photoplethysmogram (PPG) waveform.

The pressure waves produced at the heart propagate through the arteries at a certain velocity known as the pulse-wave velocity. This velocity depends on the elastic properties of arteries and blood. The Moens-Korteweg equation provides the pulse wave velocity as a function of vessel and fluid characteristics:

$$c = \frac{1}{\sqrt{ρ ( \frac{1}{ɛ} + \frac{2 R}{E \cdot h} )}} \approx \sqrt{\frac{E \cdot h}{ρ \cdot 2 R}} , \qquad ( 1 )$$

where c is the wave velocity, ε is the modulus of fluid elasticity, ρ is the fluid density, R is the inner radius of the vessel, E is the modulus of wall elasticity (i.e., Young modulus), and h is the vessel thickness.

The wave velocity can be related to the length of the vessel L and the time it takes for a pressure pulse to transit through that length, also known as the Pulse Transit Time (PTT):

$$c = \frac{L}{PTT} . \qquad ( 2 )$$

For an elastic vessel, an empirical exponential relation between the modulus E and the fluid pressure P may be derived as:

E=E₀e^{α(P-Pis 0}⁾, (3)

where E₀ and P₀ are nominal values of Young modulus and pressure, respectively, and α is a constant.

A logarithmic relation between the blood pressure, the fluid pressure P and the PTT may be obtained from equations (1) and (2) as:

BP=a₁ log PTT+b₁, (4)

where a₁ and b₁ are constants, and a₁ is typically a negative value.

A model for BP estimation can be derived if small changes of the pressure P around P₀ are assumed, and using the approximation of equation (3) as E≈E₀(1+α(P−P₀)). Then, the inverse quadratic relation between the BP and the PTT may be derived as:

$$BP = {a}_{2} \cdot \frac{1}{{PTT}^{2}} + {b}_{2} . \qquad ( 5 )$$

Another BP estimation model from the prior art assumes the inverse linear relation between the BP and the PTT:

$$BP = {a}_{3} \cdot \frac{1}{PTT} + {b}_{3} . \qquad ( 6 )$$

Yet another BP estimation model can be derived for small changes of PTT around a nominal value PTT₀. Linearization of equation (4) can lead to a following model:

BP=a₄·PTT+b₄, (7)

where a₄ and b₄ are appropriate constants.

Association for the Advancement of Medical Instrumentation (AAMI) requirements for BP estimation indicate that the mean of estimation error has to be lower than 5 mmHg in absolute value, and that the standard deviation of estimation error has to be below 8 mmHg, both for SBP and DBP. The compact notation |5|±8 mmHg can be used to denote this requirement.

For the estimation method defined by equation (7), combination of measurements and estimation can be used to obtain the BP estimate. In the prior art, the estimation method given by equation (4) was used to estimate DBP, while the difference between SBP and DBP can be determined based on equation (5). Using this method, the SBP and DBP estimates can be within 0.6±9.8 mmHg and 0.9±5.6 mmHg, respectively.

Equations (4)-(6) were used in the prior art to determine the SBP, while the PTT was replaced by the measured PAT. The root mean square error obtained when using measurements of SBP and PAT was between 4.4 mmHg and 7.5 mmHg, while performing training and testing on the same data.

An affine relation between the SBP and PTT is also observed in the prior art. Using measurements of PTT, estimates of DBP and SBP are obtained with standard deviation of error of 4.3 mmHg and 6.6 mmHg, respectively, while performing training and testing on the same data. It has been also determined that a Pre-Ejection Period (PEP) has a significant contribution on the PAT, generally more than the PTT. Strong correlations are also found between the SBP and the PAT, as well as between the SBP and the PEP.

An important issue for estimating blood pressure from the PAT is calibration, i.e., model training. In general, different patients do not have the same calibration parameters, and therefore per-patient calibration can be required to obtain accurate BP estimates. Moreover, strong correlations between the BP and the PAT have been observed whenever the window between calibration and estimation is short, while the correlation decreases for a longer window between calibration and estimation. It is suggested in the prior art that the relation between the BP and the PPG waveform is stable only within 20 minutes, and periodic calibration is required to obtain acceptable estimation results.

Previous work in the art has been targeted at estimating blood pressure from the PAT only (i.e., from the PTT). Both the PAT and the heart rate provide significant correlation with the BP, especially if multiple patients are considered. The BP estimation from the PAT alone can limit estimation accuracy. Therefore, there is a need in the art for a method of blood pressure estimation with improved accuracy, while a non-invasive technique is used.

### SUMMARY

Certain aspects provide a method for estimating a blood pressure. The method generally includes estimating a pulse arrival time (PAT), estimating a heart rate (HR), and estimating the blood pressure based on the estimated PAT and HR.

Certain aspects provide an apparatus for estimating a blood pressure. The apparatus generally includes a first estimating circuit configured to estimate a pulse arrival time (PAT), a second estimating circuit configured to estimate a heart rate (HR), and a third estimating circuit configured to estimate the blood pressure based on the estimated PAT and HR.

Certain aspects provide an apparatus for estimating a blood pressure. The apparatus generally includes means for estimating a pulse arrival time (PAT), means for estimating a heart rate (HR), and means for estimating the blood pressure based on the estimated PAT and HR.

Certain aspects provide a computer-program product for estimating a blood pressure. The computer-program product includes a computer-readable medium comprising instructions executable to estimate a pulse arrival time (PAT), estimate a heart rate (HR), and estimate the blood pressure based on the estimated PAT and HR.

Certain aspects provide a medical sensing device. The medical sensing device generally includes a sensor configured to sense a plurality of signals, a first estimating circuit configured to estimate a pulse arrival time (PAT) based on at least two of the sensed signals, a second estimating circuit configured to estimate a heart rate (HR) based on at least one of the sensed signals, a third estimating circuit configured to estimate a blood pressure based on the estimated PAT and HR, and a user interface configured to provide an indication based on the estimated blood pressure.

Certain aspects provide a watch. The watch generally includes a first estimating circuit configured to estimate a pulse arrival time (PAT), a second estimating circuit configured to estimate a heart rate (HR), a third estimating circuit configured to estimate a blood pressure based on the estimated PAT and HR, and a user interface configured to provide an indication based on the estimated blood pressure.

Certain aspects provide a monitoring device. The monitoring device generally includes a connector, a receiver configured to receive via the connector a plurality of signals, a first estimating circuit configured to estimate a pulse arrival time (PAT) based on at least two of the received signals, a second estimating circuit configured to estimate a heart rate (HR) based on at least one of the received signals, a third estimating circuit configured to estimate a blood pressure based on the estimated PAT and HR, and a user interface configured to provide an indication based on the estimated blood pressure.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 illustrates an example wireless communication system, in accordance with certain aspects of the present disclosure.

FIG. 2 illustrates various components that may be utilized in a wireless device in accordance with certain aspects of the present disclosure.

FIG. 3 illustrates an example transmitter and an example receiver that may be used within a wireless communication system in accordance with certain aspects of the present disclosure.

FIG. 4 illustrates an example of a body area network (BAN) in accordance with certain aspects of the present disclosure.

FIG. 5 illustrates an example of a pulse arrival time (PAT) measured between R peak of an electro-cardiogram (ECG) signal and several points of a photoplethysmograph (PPG) signal in accordance with certain aspects of the present disclosure.

FIG. 6 illustrates sample waveforms from a multi-parameter intelligent monitoring for intensive care (MIMIC) database in accordance with certain aspects of the present disclosure.

FIG. 7 illustrates example operations for blood pressure (BP) estimation in accordance with certain aspects of the present disclosure.

FIG. 7A illustrates example components capable of performing the operations illustrated in FIG. 7.

FIG. 8 illustrates example operations for blood pressure (BP) estimation with adaptive calibration in accordance with certain aspects of the present disclosure.

FIG. 8A illustrates example components capable of performing the operations illustrated in FIG. 8.

FIG. 9 illustrates an example of error mean, error standard deviation and mean square error for different BP estimation algorithms averaged over patient records in accordance with certain aspects of the present disclosure.

FIG. 10 illustrates an example of standard deviation of systolic blood pressure (SBP) and diastolic blood pressure (DBP) estimation errors for different patient records and different algorithms in accordance with certain aspects of the present disclosure.

FIG. 11 illustrates example histograms of means and standard deviations of estimation errors for SBP and DBP in accordance with certain aspects of the present disclosure.

FIG. 12 illustrates an example of actual and estimated SBP and DBP in accordance with certain aspects of the present disclosure.

FIG. 13 illustrates an example standard deviation of SBP error versus calibration period in accordance with certain aspects of the present disclosure.

FIG. 14 illustrates an example standard deviation of SBP and DBP error versus artificially added PAT jitter in accordance with certain aspects of the present disclosure.

FIG. 15 illustrates an example standard deviation of SBP and DBP error versus artificially added PAT skew in accordance with certain aspects of the present disclosure.

FIG. 16 illustrates an example absolute mean of SBP and DBP error versus artificially added PAT skew in accordance with certain aspects of the present disclosure.

### DETAILED DESCRIPTION

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different wireless technologies, system configurations, networks, and transmission protocols, some of which are illustrated by way of example in the figures and in the following description of the preferred aspects. The detailed description and drawings are merely illustrative of the disclosure rather than limiting, the scope of the disclosure being defined by the appended claims and equivalents thereof.

### An Example Wireless Communication System

The techniques described herein may be used for various broadband wireless communication systems, including communication systems that are based on an orthogonal multiplexing scheme and a single carrier transmission. Examples of such communication systems include Orthogonal Frequency Division Multiple Access (OFDMA) systems, Single-Carrier Frequency Division Multiple Access (SC-FDMA) systems, Code Division Multiple Access (CDMA), and so forth. An OFDMA system utilizes orthogonal frequency division multiplexing (OFDM), which is a modulation technique that partitions the overall system bandwidth into multiple orthogonal sub-carriers. These sub-carriers may also be called tones, bins, etc. With OFDM, each sub-carrier may be independently modulated with data. An SC-FDMA system may utilize interleaved FDMA (IFDMA) to transmit on sub-carriers that are distributed across the system bandwidth, localized FDMA (LFDMA) to transmit on a block of adjacent sub-carriers, or enhanced FDMA (EFDMA) to transmit on multiple blocks of adjacent sub-carriers. In general, modulation symbols are sent in the frequency domain with OFDM and in the time domain with SC-FDMA. A CDMA system may utilize spread-spectrum technology and a coding scheme where each transmitter (i.e., user) is assigned a code in order to allow multiple users to be multiplexed over the same physical channel.

One specific example of a communication system based on an orthogonal multiplexing scheme is a WiMAX system. WiMAX, which stands for the Worldwide Interoperability for Microwave Access, is a standards-based broadband wireless technology that provides high-throughput broadband connections over long distances. There are two main applications of WiMAX today: fixed WiMAX and mobile WiMAX. Fixed WiMAX applications are point-to-multipoint, enabling broadband access to homes and businesses, for example. Mobile WiMAX offers the full mobility of cellular networks at broadband speeds.

IEEE 802.16x is an emerging standard organization to define an air interface for fixed and mobile broadband wireless access (BWA) systems. IEEE 802.16x approved “IEEE P802.16d/D5-2004” in May 2004 for fixed BWA systems and published “IEEE P802.16e/D12 Oct. 2005” in October 2005 for mobile BWA systems. The latest revision of the IEEE 802.16, “IEEE P802.16Rev2/D8 December 2008”, a draft standard, now consolidates materials from IEEE 802.16e and corrigendum. The standards define four different physical layers (PHYs) and one medium access control (MAC) layer. The OFDM and OFDMA physical layer of the four physical layers are the most popular in the fixed and mobile BWA areas respectively.

The teachings herein may be incorporated into (e.g., implemented within or performed by) a variety of wired or wireless apparatuses (e.g., nodes). In some aspects, a node implemented in accordance with the teachings herein may comprise an access point or an access terminal.

An access terminal (“AT”) may comprise, be implemented as, or known as an access terminal, a subscriber station, a subscriber unit, a mobile station, a remote station, a remote terminal, a user terminal, a user agent, a user device, user equipment, or some other terminology. In some implementations an access terminal may comprise a cellular telephone, a cordless telephone, a Session Initiation Protocol (“SIP”) phone, a wireless local loop (“WLL”) station, a personal digital assistant (“PDA”), a handheld device having wireless connection capability, or some other suitable processing device connected to a wireless modem. Accordingly, one or more aspects taught herein may be incorporated into a phone (e.g., a cellular phone or smart phone), a computer (e.g., a laptop), a portable communication device, a portable computing device (e.g., a personal data assistant), an entertainment device (e.g., a music or video device, or a satellite radio), a global positioning system device, or any other suitable device that is configured to communicate via a wireless or wired medium. In some aspects the node is a wireless node. Such wireless nodes may provide, for example, connectivity for or to a network (e.g., a wide area network such as the Internet or a cellular network) via a wired or wireless communication link.

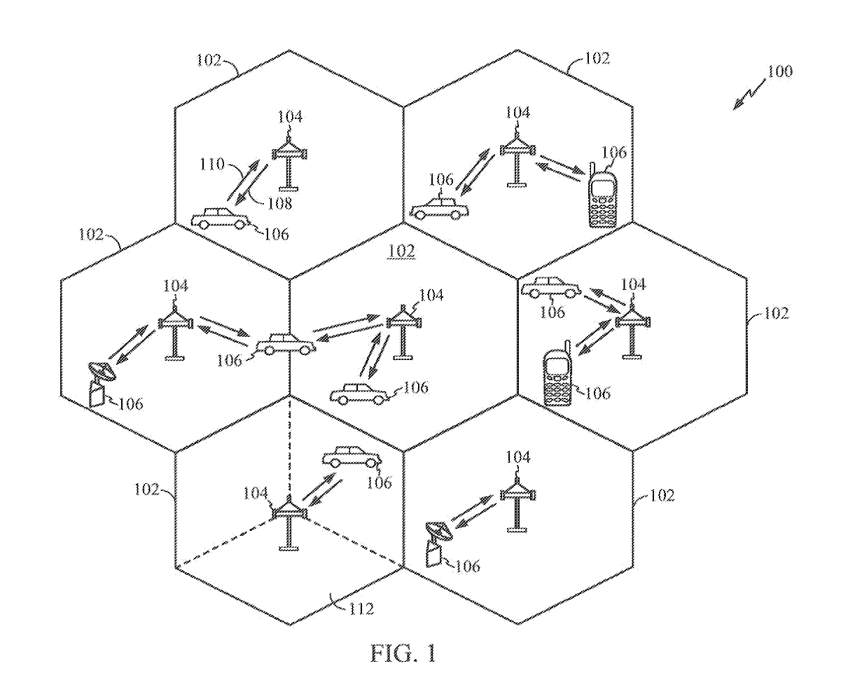

FIG. 1 illustrates an example of a wireless communication system 100 in which aspects of the present disclosure may be employed. The wireless communication system 100 may be a broadband wireless communication system. The wireless communication system 100 may provide communication for a number of cells 102, each of which is serviced by a base station 104. A base station 104 may be a fixed station that communicates with user terminals 106. The base station 104 may alternatively be referred to as an access point, a Node B or some other terminology.

FIG. 1 depicts various user terminals 106 dispersed throughout the system 100. The user terminals 106 may be fixed (i.e., stationary) or mobile. The user terminals 106 may alternatively be referred to as remote stations, access terminals, terminals, subscriber units, mobile stations, stations, user equipment, etc. The user terminals 106 may be wireless devices, such as cellular phones, personal digital assistants (PDAs), handheld devices, wireless modems, laptop computers, personal computers, etc.

A variety of algorithms and methods may be used for transmissions in the wireless communication system 100 between the base stations 104 and the user terminals 106. For example, signals may be sent and received between the base stations 104 and the user terminals 106 in accordance with OFDM/OFDMA techniques. If this is the case, the wireless communication system 100 may be referred to as an OFDM/OFDMA system. Alternatively, signals may be sent and received between the base stations 104 and the user terminals 106 in accordance with CDMA technique. If this is the case, the wireless communication system 100 may be referred to as a CDMA system.

A communication link that facilitates transmission from a base station 104 to a user terminal 106 may be referred to as a downlink (DL) 108, and a communication link that facilitates transmission from a user terminal 106 to a base station 104 may be referred to as an uplink (UL) 110. Alternatively, a downlink 108 may be referred to as a forward link or a forward channel, and an uplink 110 may be referred to as a reverse link or a reverse channel.

A cell 102 may be divided into multiple sectors 112. A sector 112 is a physical coverage area within a cell 102. Base stations 104 within a wireless communication system 100 may utilize antennas that concentrate the flow of power within a particular sector 112 of the cell 102. Such antennas may be referred to as directional antennas.

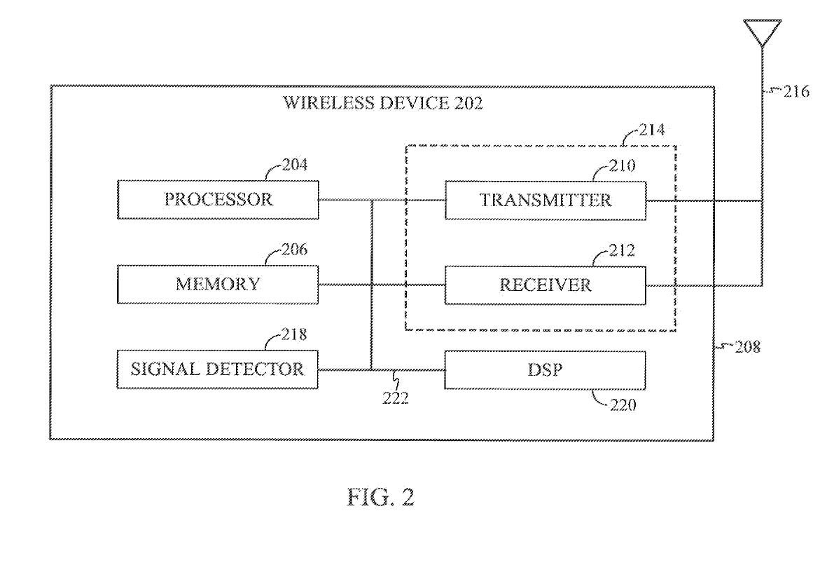

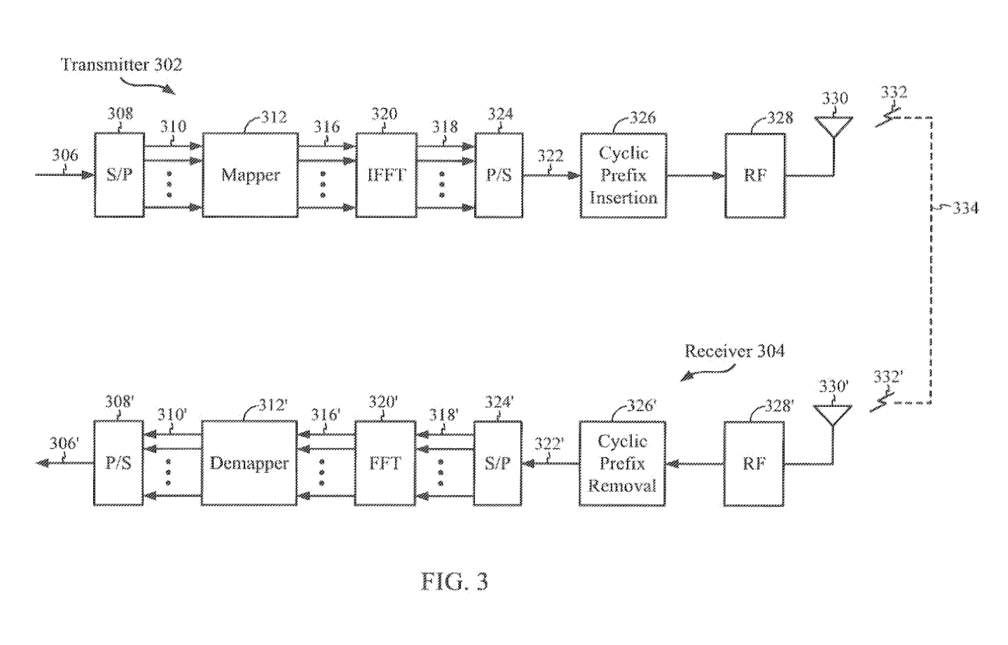

FIG. 2 illustrates various components that may be utilized in a wireless device 202 that may be employed within the wireless communication system 100. The wireless device 202 is an example of a device that may be configured to implement the various methods described herein. The wireless device 202 may be a base station 104 or a user terminal 106.

The wireless device 202 may include a processor 204 which controls operation of the wireless device 202. The processor 204 may also be referred to as a central processing unit (CPU). Memory 206, which may include both read-only memory (ROM) and random access memory (RAM), provides instructions and data to the processor 204. A portion of the memory 206 may also include non-volatile random access memory (NVRAM). The processor 204 typically performs logical and arithmetic operations based on program instructions stored within the memory 206. The instructions in the memory 206 may be executable to implement the methods described herein.

The wireless device 202 may also include a housing 208 that may include a transmitter 210 and a receiver 212 to allow transmission and reception of data between the wireless device 202 and a remote location. The transmitter 210 and receiver 212 may be combined into a transceiver 214. An antenna 216 may be attached to the housing 208 and electrically coupled to the transceiver 214. The wireless device 202 may also include (not shown) multiple transmitters, multiple receivers, multiple transceivers, and/or multiple antennas.

The wireless device 202 may also include a signal detector 218 that may be used in an effort to detect and quantify the level of signals received by the transceiver 214. The signal detector 218 may detect such signals as total energy, energy per subcarrier per symbol, power spectral density and other signals. The wireless device 202 may also include a digital signal processor (DSP) 220 for use in processing signals.

The various components of the wireless device 202 may be coupled together by a bus system 222, which may include a power bus, a control signal bus, and a status signal bus in addition to a data bus.

FIG. 3 illustrates an example of a transmitter 302 that may be used within a wireless communication system 100 that utilizes OFDM/OFDMA. Portions of the transmitter 302 may be implemented in the transmitter 210 of a wireless device 202. The transmitter 302 may be implemented in a base station 104 for transmitting data 306 to a user terminal 106 on a downlink 108. The transmitter 302 may also be implemented in a user terminal 106 for transmitting data 306 to a base station 104 on an uplink 110.

Data 306 to be transmitted is shown being provided as input to a serial-to-parallel (S/P) converter 308. The S/P converter 308 may split the transmission data into M parallel data streams 310.

The N parallel data streams 310 may then be provided as input to a mapper 312. The mapper 312 may map the N parallel data streams 310 onto N constellation points. The mapping may be done using some modulation constellation, such as binary phase-shift keying (BPSK), quadrature phase-shift keying (QPSK), 8 phase-shift keying (8PSK), quadrature amplitude modulation (QAM), etc. Thus, the mapper 312 may output N parallel symbol streams 316, each symbol stream 316 corresponding to one of the N orthogonal subcarriers of the inverse fast Fourier transform (IFFT) 320. These N parallel symbol streams 316 are represented in the frequency domain and may be converted into N parallel time domain sample streams 318 by an IFFT component 320.

A brief note about terminology will now be provided. N parallel modulations in the frequency domain are equal to N modulation symbols in the frequency domain, which are equal to N mapping and N-point IFFT in the frequency domain, which is equal to one (useful) OFDM symbol in the time domain, which is equal to N samples in the time domain. One OFDM symbol in the time domain, N_{S}, is equal to N_{CP} (the number of cyclic prefix (CP) samples per OFDM symbol)+N (the number of useful samples per OFDM symbol).

The N parallel time domain sample streams 318 may be converted into an OFDM/OFDMA symbol stream 322 by a parallel-to-serial (P/S) converter 324. A cyclic prefix insertion component 326 may insert a CP between successive OFDM/OFDMA symbols in the OFDM/OFDMA symbol stream 322. The output of the CP insertion component 326 may then be upconverted to a desired transmit frequency band by a radio frequency (RF) front end 328. An antenna 330 may then transmit the resulting signal 332.

FIG. 3 also illustrates an example of a receiver 304 that may be used within a wireless device 202 that utilizes OFDM/OFDMA. Portions of the receiver 304 may be implemented in the receiver 212 of a wireless device 202. The receiver 304 may be implemented in a user terminal 106 for receiving data 306 from a base station 104 on a downlink 108. The receiver 304 may also be implemented in a base station 104 for receiving data 306 from a user terminal 106 on an uplink 110.

The transmitted signal 332 is shown traveling over a wireless channel 334. When a signal 332′ is received by an antenna 330′, the received signal 332′ may be downconverted to a baseband signal by an RF front end 328′. A CP removal component 326′ may then remove the CP that was inserted between OFDM/OFDMA symbols by the CP insertion component 326.

The output of the CP removal component 326′ may be provided to an S/P converter 324′. The S/P converter 324′ may divide the OFDM/OFDMA symbol stream 322′ into the N parallel time-domain symbol streams 318′, each of which corresponds to one of the N orthogonal subcarriers. A fast Fourier transform (FFT) component 320′ may convert the N parallel time-domain symbol streams 318′ into the frequency domain and output N parallel frequency-domain symbol streams 316′.

A demapper 312′ may perform the inverse of the symbol mapping operation that was performed by the mapper 312 thereby outputting N parallel data streams 310′. A P/S converter 308′ may combine the N parallel data streams 310′ into a single data stream 306′. Ideally, this data stream 306′ corresponds to the data 306 that was provided as input to the transmitter 302. Note that elements 308′, 310′, 312′, 316′, 320′, 318′ and 324′ may all be found in a baseband processor 340′.

### Body Area Network Concept

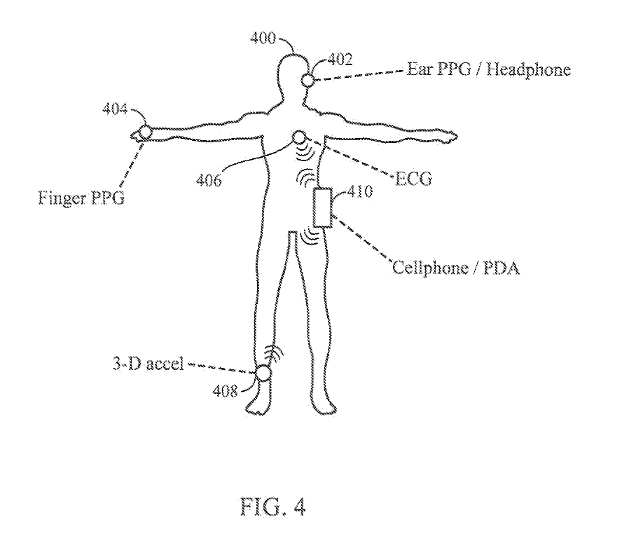

FIG. 4 illustrates an example of a body area network (BAN) 400 that may correspond to the wireless system 100 illustrated in FIG. 1. Body area networks represent a promising concept for healthcare applications such as continuous monitoring for diagnostic purposes, effects of medicines on chronic ailments, etc.

The BAN may consist of several acquisition circuits as illustrated in FIG. 4. Each acquisition circuit may comprise wireless sensor that senses one or more vital signs and communicates them to an aggregator (i.e., an access terminal) such as a mobile handset, a wireless watch, or a Personal Data Assistant (PDA). Sensors 402, 404, 406, and 408 may acquire various biomedical signals and transmit them over a wireless channel to an aggregator 410. The sensors 402-408 may have the same functionality as access points 104 illustrated in FIG. 1.

The aggregator 410 illustrated in FIG. 4 may receive and process various biomedical signals transmitted over a wireless channel from the sensors 402-408. The aggregator 410 may be a mobile handset or a PDA, and may have the same functionality as a mobile device 106 from FIG. 1.

The raw sensor measurements and/or their processed versions can be of use for diagnostic purposes. These measurements can also be used to evaluate short and long term effectiveness of drugs and therapy. In such application, the sensors may need to be small, lightweight, have long battery life and low cost. This results into very low power requirements for sensing and communicating, as well as into low complexity processing at the nodes.

Blood pressure monitoring techniques that avoid inflating or wearing a cuff can be very attractive for low-power applications. In the present disclosure, electrocardiogram (ECG) and photoplethysmogram (PPG) signals may be measured, both of which may be obtained non-invasively utilizing low-power and low-cost electronics. The measured ECG and PPG signal may be then used for computing a pulse arrival time (PAT) and for estimating the blood pressure. An important issue addressed in the present disclosure is also synchronization (i.e., skew requirement) among wireless sensors in the BAN for accurate estimation of the PAT.

Certain aspects of the present disclosure support utilizing the heart rate (HR) measurements along with ECG and PPG peaks in order to improve accuracy of blood pressure estimation. The estimation model may be trained to utilize the HR, ECG and PPG signals. A recursive least squares estimation may be applied to improve adaptation of the estimation model. The adapted estimation model may be further conditioned to improve robustness. Certain aspects of the present disclosure support additional means to correct bias terms after adaptation. The resulted estimation model provides improved estimation accuracy and robustness, while being compliant with the AAMI accuracy requirements for longer durations before requiring re-calibration of model parameters.

### Overview of the Proposed Estimation Method

Certain aspects of the present disclosure support estimating SBP and DBP from a combination of PAT and heart rate (HR) signals. Related prior art described above has been targeted at estimating BP from PAT only, but not from both PAT and HR. It can be observed that both quantities provide significant correlation with BP, especially when multiple patients are considered. This may improve accuracy of estimating BP, compared with estimating BP from the PAT only or from the HR only.

Certain aspects of the present disclosure support initial training and subsequent retraining of data using the adaptive recursive least-square (RLS) filtering approach. On the other hand, calibration algorithms in the prior art are typically based on “single-point” training.

The MIMIC database can be used for simulation purposes. The MIMIC database contains data from several subjects and spanning several hours for each patient. Therefore, the proposed estimation method can be realistically evaluated over a diverse population and over extended periods of time.

### Measuring Pulse Arrival Time and Pulse Transit Time

A Pulse Transit Time (PTT) may be measured indirectly through a related quantity known as a Pulse Arrival Time (PAT). The PAT may capture a time that takes for a blood pressure wave to travel from the heart to a certain point in the body, such as the fingertip or earlobe. The PAT signal may be calculated from two signals: the electrocardiogram (ECG) signal and the photoplethysmogram (PPG) signal obtained at the fingertip or earlobe using a pulse oximetry sensor. The ECG signal is used to find the time where the pressure wave is generated, whereas the PPG signal is used to find the time where the pressure wave reaches the PPG sensor location.

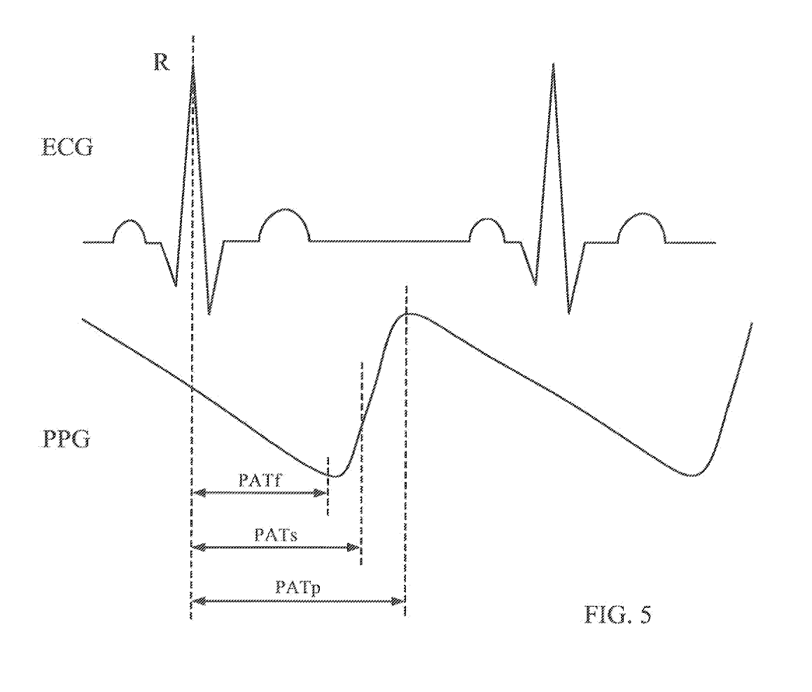

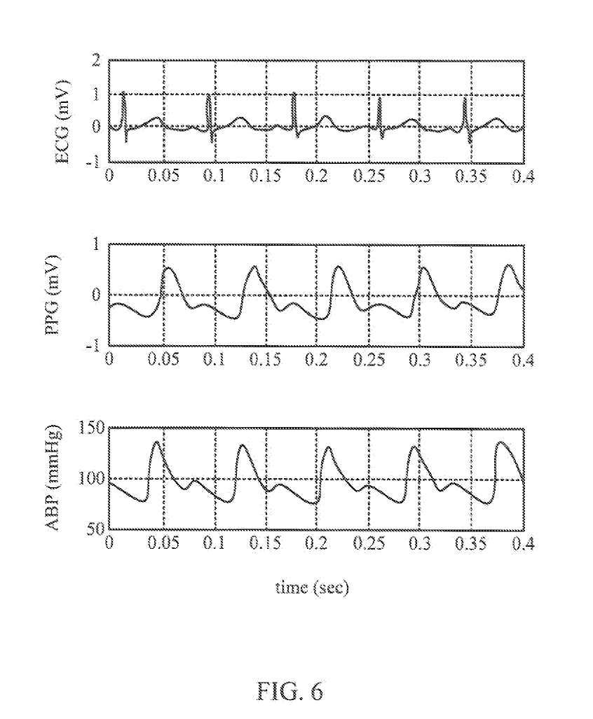

Specifically, the PAT may be measured as the delay between a particular point of the ECG signal (e.g., the R peak of the ECG signal), and a particular point in the photoplethysmogram (PPG) signal, such as the foot (PATf), peak (PATp) or the maximum slope point (PATs), as illustrated in FIG. 5. In particular, the R peak of the ECG indicates when the isovolumetric contraction of the heart starts, and the PPG signal gives the change in blood oxygenation at the measuring location. FIG. 6 illustrates sample waveforms for ECG, PPG and arterial blood pressure (ABP) signals obtained from a multi-parameter intelligent monitoring for intensive care (MIMIC) database.

In one aspect of the present disclosure, at least one of: P peak, Q peak, R peak, S peak, or T peak of the ECG signal along with the defined point of the PPG signal may be used for measuring the PAT. In another aspect, instead of the ECG signal, an acoustic signal (e.g., a heart sound) may be utilized for measuring the PAT. On the other hand, a heart rate (HR) may be measured as a time period between two consecutive peaks of the ECG signal, or of the PPG signal, or of an acoustic signal such as the heart sound.

When the PAT is measured between the peak of ECG signal and some point of the PPG signal, it may be related to the PTT as follows:

PAT=PEP+PTT, (8)

where PEP is a Pre-Ejection Period representing the isovolumetric contraction time of the heart, which is the time that takes for the myocardium to raise enough pressure to open the aortic valve and to start pushing blood out of the ventricle. On the other hand, the PTT represents the time it takes for the blood pressure wave to travel from the heart to the place where the PPG is being sensed.

The PTT may be inversely proportional to a wave velocity, whereas the PEP is not related to it. Thus, only the PTT is related to the BP through equation (1). The effect of the PEP on the PAT can be considerable, and therefore the PAT may be unreliable for estimating the BP. Nonetheless, there is also strong correlation between the BP and the PAT. Moreover, employing the ECG can be attractive for BANs since it requires a low power instrumentation amplifier to obtain it, and it is comparatively robust to movement and other artifacts.

### Segmentation of ECG, PPG and ABP Signals

Certain aspects of the present disclosure support estimating BP from ECG and PPG signals. In order to provide accurate estimate, the exact location of the R peak of the ECG signal may need to be found. In order to compute PATp, PATf and PATs values, it may be also required to find the peak, foot and maximum slope point, respectively, of the PPG signal. Moreover, it may be required to segment the signals to obtain the peak and foot in order to obtain SBP and DBP values. FIG. 5 illustrates an example of the PAT measured between R peak of the ECG signal and several points of the PPG signal.

The ECG segmentation may be accomplished in three steps. First, the ECG signal may be band-pass filtered between 8 Hz and 15 Hz. The resulting signal may be squared and processed in segments of variable duration. The initial segment duration may be, for example, 2 seconds. For every segment, a threshold may be computed and all the peaks above the threshold may be located. Then, for example, all peaks that are less than 0.17 seconds apart may be removed, while always maintaining the peak with the highest amplitude.

Subsequently, a tracking condition may be applied to determine the average period of the ECG signal. The tracking condition may be defined as follows. Let

$$\overline{p} = \frac{1}{N} {\sum }_{i = 1}^{N} p ( i ) and {σ}^{2} = \frac{1}{N} {\sum }_{i = 1}^{N} {( p ( i ) - \overline{p} )}^{2} , \qquad ( 9 )$$

where p(i), i=1, . . . , N represents a sequence of N consecutive peaks (e.g., N=5), \ddot{p} represents a mean of the sequence p(i) and σ² is a standard deviation of the sequence p(i).

The tracking condition may be now defined as:

Tracking condition: σ<0.2 p̅. (10)

Thus, if the differences between the locations of the last five peaks have a standard deviation of less than 20% of their mean, then a regular period may be detected and the segment length may be set to twice that period. Therefore, an adaptive thresholding mechanism is implicit in this operation. Furthermore, all peaks which are less than half the regular period apart may be also removed.

The locations of the peaks detected from the band-pass filtered signal may be finally corrected by looking at the closest maximum point in the original (unfiltered) ECG signal and within a window of duration ±20% of the regular period. Thus, it can be expected that the R peak has higher amplitude than any other point in the ECG waveform within that window. This may not be generally true since it is observed that some subjects may have a T-wave with higher amplitude than the R peak, as well as close in time to it. However, for most subjects the R peak has higher amplitude than any other point in each heart beat. Therefore, this condition may be required to accurately find the location of the R peaks.

In order to segment the PPG signal, the segment between two consecutive ECG peaks may be analyzed. The peaks and feet (valleys) may be detected by finding the maximum and minimum points within this segment. The maximum slope point may be detected by finding the point with maximum difference, while searching between a foot and a subsequent peak. The tracking condition given by equation (10) may be applied on the PPG peaks, PPG maximum slope points, and PPG feet, while only those points where the tracking condition is met in all cases may be kept. If this is the case, then the delay r may be computed between the two consecutive ECG peaks, and the instantaneous heart rate may be computed as HR=60/τ (beats per minute).

### Method for Blood Pressure Estimation

Certain aspects of the present disclosure support estimating a blood pressure (BP) based on a heart rate (HR) and on a pulse arrival time (PAT). A linear model may be used to estimate the BP from the PAT and HR. The model may be given as:

SBP=a₁·PAT+b₁·HR+c₁,

DBP=a₂·PAT+b₂·HR+c₂ (11)

where aᵢ, bᵢ and cᵢ, i=1, 2 are constants to be determined. The model defined by equation (11) is based on the model defined by equation (5), as well as on the observation that the BP increases with the HR. It should be however noted that constraints for determining unknown constants in equation (11) are different from constraints applied in equation (5).

Model training may be obtained through a least-squares procedure. The unknown parameters from equation (11) may be grouped into an unknown parameter matrix θ such that:

$$θ = [ \begin{matrix}{a}_{1} & {a}_{2} \\ {b}_{1} & {b}_{2} \\ {c}_{1} & {c}_{2}\end{matrix} ] . \qquad ( 12 )$$

N observations SBP(i), DBP(i), PAT(i), HR(i) from time instants i=i₁, . . . , i_{N} may be available. These observations may be collected into matrices as:

$$\begin{matrix}{Y}_{1 \text{:} N} = [ \begin{matrix}SBP ( {i}_{1} ) & DBP ( {i}_{1} ) \\ ⋮ & ⋮ \\ SBP ( {i}_{N} ) & DBP ( {i}_{N} )\end{matrix} ] , & ( 13 ) \\ {X}_{1 \text{:} N} = [ \begin{matrix}PAT ( {i}_{1} ) & HR ( {i}_{1} ) & 1 \\ ⋮ & ⋮ & ⋮ \\ PAT ( {i}_{N} ) & HR ( {i}_{N} ) & 1\end{matrix} ] . & ( 14 )\end{matrix}$$

Then, the estimate of the parameter matrix θ that minimizes squared error ∥Y_{1:N}−X_{1:N}·θ∥² may be given as:

θ_{N}[X_{1:N}*X_{1:N}]⁻¹·X_{1:N}*·Y_{1:N}, (15)

where the operator * denotes conjugate transposition. Given a new set of measurements X, an estimate of Y denoted by Ŷ may be obtained as follows:

Ŷ=X·θ_{N}. (16)

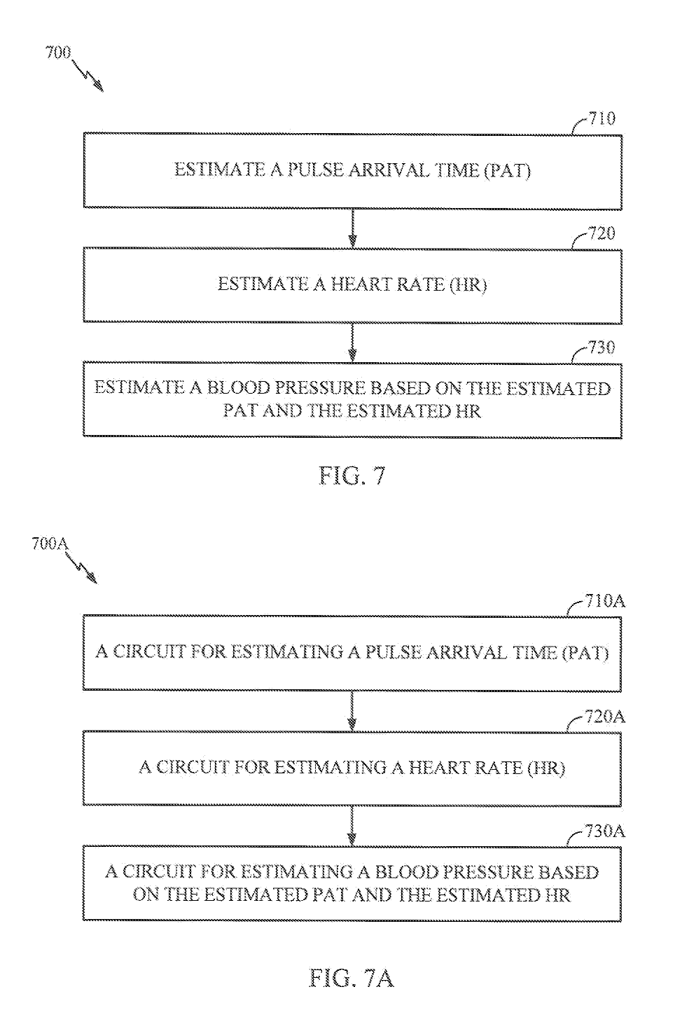

FIG. 7 illustrates example operations 700 for BP estimation in accordance with certain aspects of the present disclosure. At 710, the PAT may be estimated, and, at 720, the HR may be estimated. The BP may be then calculated, at 730, based on the estimated PAT, the estimated HR and the estimated model parameters according to the equations (15)-(16).

### Adaptive Calibration of Model Parameters for Blood Pressure Estimation

Calibration of the parameters from the estimation model defined by equation (11) may have two aspects. First, initial per-user calibration may be required, given a certain number of measurements of SBP and DBP. This would correspond to the case when the subject uses for the first time the system for BP estimation, where the initial BP measurements may be taken using a standard procedure (e.g., the ausculatory or oscillometric procedure). Here, the terms “measurements” and “observations” can be used synonymously. The number of SBP and DBP observations to be utilized may be in the order of 10 to 40. Once these observations are available, then equation (15) may be applied to obtain an initial estimate of the parameter matrix θ defined by equation (12). The initial calibration instants can be denoted as i₁, . . . , i_{N}, and the parameter estimates after calibration can be denoted as θ_{N}. Also, P_{N} denotes the inverse of the correlation matrix of the initial estimate, i.e.,

P_{N}=[X_{1:N}*·X_{1:N}]⁻¹. (17)

The estimation performance may remain accurate within a certain period after calibration, and re-calibration may be required after this period. It can be assumed that the re-calibration requires the user to take one measurement of SBP and DBP using, for example, a cuff-based oscillometric device available for home use. Let T_{cal} denote the time interval between consecutive calibration instances, and i_{N+1} denote the first re-calibration instant after the initial calibration. It is important to note that i_{N} and i_{N+1} may be arbitrary time instants, while not being contiguous. Then, given a new set of observations SBP(i_{N+1}), DBP(i_{N+1}), PAT(i_{N+1}) and HR(i_{N+1}), these new observations may be incorporated into the least-squares solution given by equation (11) by using the RLS algorithmic recursions. That is, the solution that minimizes ∥Y_{1:N+1}−X_{1:N+1}·θ∥² may be recursively found as:

$$\begin{matrix}{θ}_{N + 1} = {θ}_{N} + \frac{{λ}^{- 1} {P}_{N} {u}_{N + 1}^{*} ( {d}_{N + 1} - {u}_{N + 1} \cdot {θ}_{N} )}{1 + {λ}^{- 1} {u}_{N} {P}_{N} {u}_{N + 1}^{*}} , & ( 18 ) \\ {P}_{N + 1} = {P}_{N} - \frac{{λ}^{- 2} {P}_{N} {u}_{N + 1}^{*} {u}_{N + 1} \cdot {P}_{N}}{1 + {λ}^{- 1} {u}_{N + 1} {P}_{N} {u}_{N + 1}^{*}} , \\ where & ( 19 ) \\ {d}_{N + 1} = [ SBP ( {i}_{N + 1} ) DBP ( {i}_{N + 1} ) ] , & ( 20 ) \\ {u}_{N + 1} = [ PAT ( {i}_{N + 1} ) HR ( {i}_{N + 1} ) 1 ] , & ( 21 )\end{matrix}$$

and λ is a forgetting factor, typically chosen as 0<<λ≦1. For example, λ=0.95 may provide good estimation accuracy.

The measurements of SBP, DBP, and estimates of PAT and HR may be considerably noisy, and thus may produce estimates of the parameter matrix θ which are not physically possible. Thus, a mechanism may be adopted to enhance robustness, whereby the parameters a₁, a₂, b₁, b₂ may be kept within certain limits. Whenever a parameter obtained through equation (15) or equations (18)-(19) is outside of the allowed range, the parameters may be rounded to the closest point in that range.

The minimum and maximum parameter values can be denoted as θₘᵢₙ and θₘₐₓ, respectively. Depending on the correlations between the observations, it may be allowed to change these particular limits. For example, the following allowed ranges for the parameters may be adopted:

$$\begin{matrix}{θ}_{0 , min} = [ \begin{matrix}- 400 & - 300 \\ 0 & 0 \\ - ∞ & - ∞\end{matrix} ] , {θ}_{0 , max} = [ \begin{matrix}0 & 0 \\ 2 & 2 \\ ∞ & ∞\end{matrix} ] , & ( 22 ) \\ {θ}_{min} = min ( {θ}_{0 , min} , ( 1 - ρ ) ⊗ {θ}_{0 , min} + ρ ⊗ {θ}_{N} ) , & ( 23 ) \\ {θ}_{max} = max ( {θ}_{0 , max} , ( 1 - ρ ) ⊗ {θ}_{0 , max} + ρ ⊗ {θ}_{N} ) , & ( 24 )\end{matrix}$$

where \textcircled{×} represents element-wise multiplication and θ_{N} represents the parameter matrix obtained after the initial calibration, and ρ represents a matrix such that:

$$ρ = [ \begin{matrix}{ρ}_{PAT} & 0 \\ {ρ}_{HR} & 0 \\ 0 & 0\end{matrix} ] , \qquad ( 25 )$$

where ρ_{PAT} is an absolute correlation coefficient between the SBP and PAT, and ρ_{HR} is an absolute correlation coefficient between the SBP and HR. These coefficients may be obtained during the initial calibration.

Thus, whenever the PAT and SBP are strongly correlated and ρ_{PAT}≈1, if the value of a₁ obtained through calibration is below the required limit, then this limit may be reduced in order to accommodate larger variations. This approach has the advantage of avoiding restriction of the parameter a₁ for those patients where a₁ is strongly correlated with SBP, and therefore should be kept unaltered. The adjusted version of the parameter matrix θ_{N+1} may be denoted as:

η_{N+1}=min(θₘₐₓ, max(θₘᵢₙ,θ_{N+1})). (26)

After the parameters have been updated as defined by equations (18)-(19) and fixed to be within their allowed ranges as given by equation (26), the parameters c₁ and c₂ from equation (12) corresponding to the bias term may need also to be adjusted. Estimated parameters c₁ and c₂ obtained by applying equations (18)-(19) may not always provide good performance, especially since the relation between SBP, DBP, PAT and HR may tend to change with time, and this change is more prominent for the bias term. Therefore, it may be reasonable to base the bias on the latest measurements, and to give less weight to the past measurements.

The bias in the parameter matrix η_{N+1} defined by equation (26) may be corrected as follows. The resulting estimate can be denoted as γ_{N+1}, and the first two rows of γ_{N+1} may be kept unmodified, i.e.,

e₁^{T}·γ_{N+1}=e₁^{T}·η_{N+1}, e₂^{T}·γ_{N+1}=e₂^{T}·η_{N+1}, (27)

where eₖ represents a vector with a unity entry in position k and with zeros elsewhere. For the last row of γ_{N+1}, the following may hold:

e₃^{T}·γ_{N+1}=α·e₃^{T}η_{N+1}+(1−α)·(d_{N+1}−ũ_{N+1}·\tilde{η}_{N+1}), (28)

where

ũ_{N+1}=[PAT(i_{N+1})HR(i_{N+1})], (29)

\tilde{η}_{N+1}=[I₂0]·η_{N+1}, (30)

and α=0.3 may be utilized.

After γ_{N+1} is obtained, the BP estimate at an arbitrary time instant k may be obtained as follows:

\tilde{d}ₖ=uₖ·γ_{N+1}. (31)

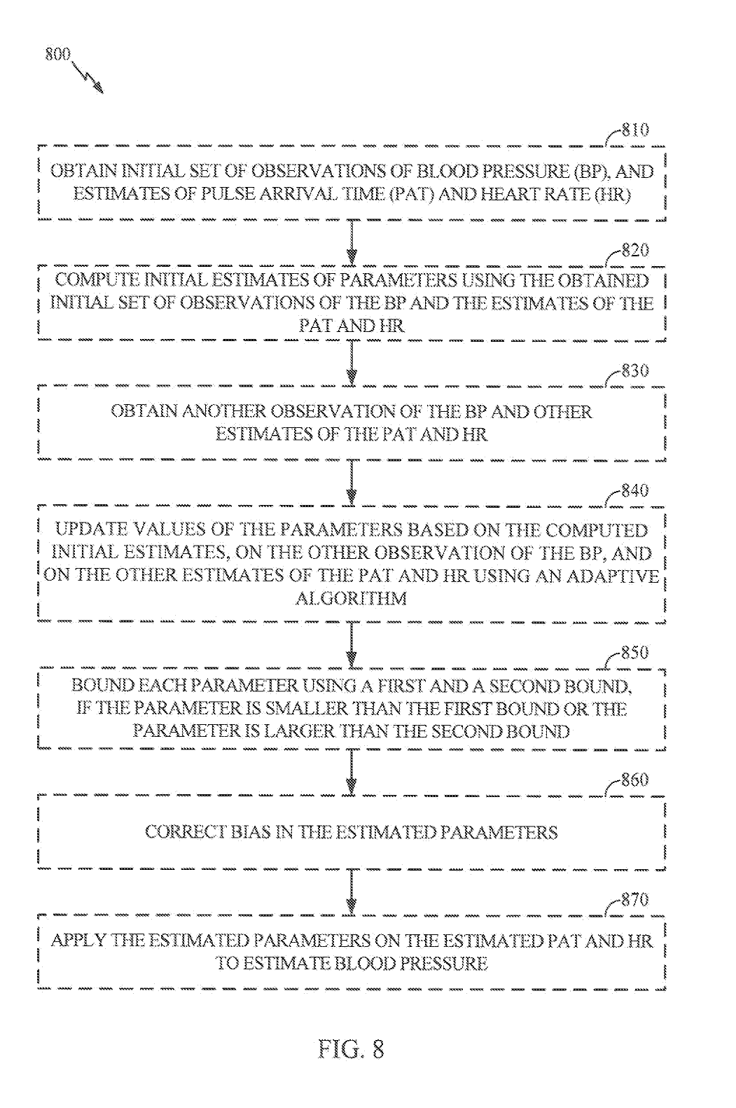

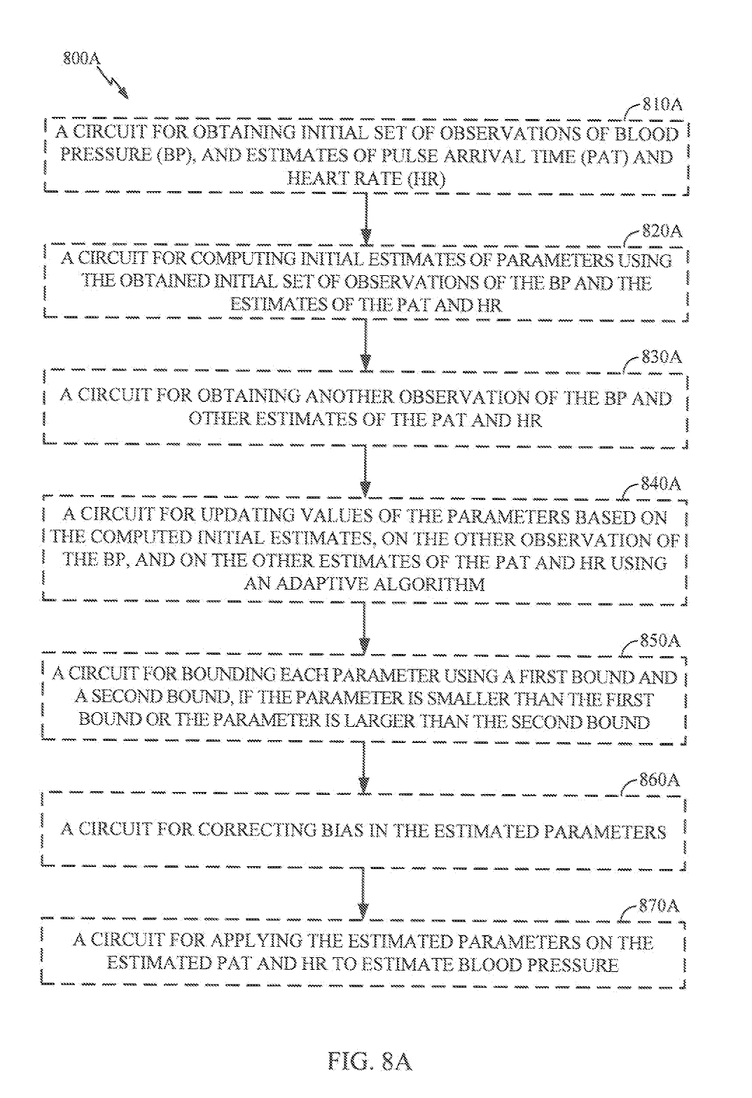

FIG. 8 summarizes operations for blood pressure (BP) estimation with adaptive calibration in accordance with certain aspects of the present disclosure. At 810, an initial set of observations of the BP and estimates of the PAT and HR may be obtained. At 820, initial set of parameters θ may be computed as given by equation (15) using the obtained initial set of observations of the BP and the estimates of the PAT and HR. At 830, another observation of the BP and other estimates of the PAT and HR may be obtained. The set of model parameters may be then updated, at 840, based on the computed initial estimates of the parameters, on the other observation of the BP, and on the other estimates of the PAT and HR using, for example, the adaptive algorithm given by equations (18)-(19).

At 850, each parameter may be bounded by using defined minimum and maximum values. These values may be, for example, given by equation (22), while the bounding algorithm may be defined by equations (23)-(26). At 860, any bias may be corrected in the estimated parameters θ, as defined by equations (27)-(30). This will produce a new parameter vector denoted by γ in equations (27)-(28). At 870, the estimated set of parameters γ may be applied on the estimated PAT and the HR in order to estimate SBP and DBP, as given by equation (31).

### Simulation Results

The proposed estimation algorithm can be applied on the MIMIC database using signals sampled at 125 Hz. All the signals are also subsequently up-sampled to 1 kHz before processing. For the initial calibration stage, 40 measurements of SBP and DBP spaced about 5 minutes apart are used. This would simulate the scenario when for the first time the user calibrates the system. Then, the proposed estimation algorithm can be applied and compared with other algorithms from the prior art. Unless otherwise noted, the parameters are re-calibrated every T_{cal}=1 hour.

Of the 72 records available in the MIMIC database, 56 of them have complete recordings of PPG, ECG and arterial blood pressure (ABP). Of these 56 records, 22 records can be removed based on the integrity of the signals. These include records with abnormal ECG waveforms where detecting the exact location of the R peak becomes difficult and error prone, together with records with extensive movement artifacts that jeopardize the integrity of either the PPG or ABP waveforms.

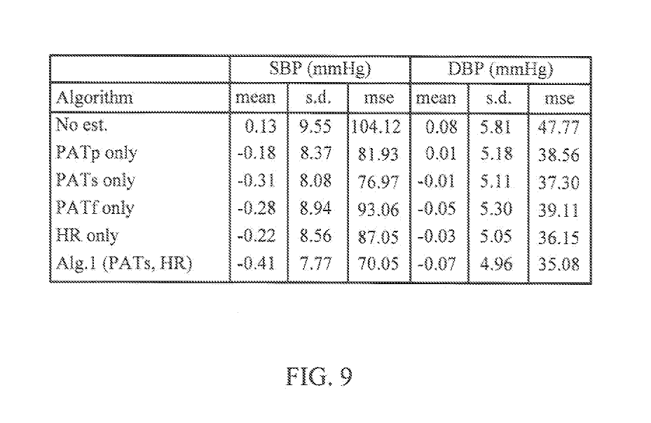

FIG. 9 illustrates the mean and standard deviation of the SBP and DBP estimation errors, averaged over all remaining 34 records from 25 different patients for T_{cal}=1 hour and for different estimation algorithms. The first algorithm, denoted by “No est.” represents a trivial estimator, where the estimated values of SBP and DBP may be equal to the measurements obtained in the latest calibration. Thus, the estimated pressure is piecewise constant. Other analyzed estimation algorithms are based on the proposed approach, but different types of measurements can be used. The algorithm denoted by “PATp only” uses only the PATp in order to perform the BP estimation, while ignoring other measurements. The coefficient range correction is also removed in this particular case. Algorithms denoted by “PATs only”, “PATf only” and “HR only” are based on the same principle as the one previously described, except that PATs, PATf and HR are respectively utilized instead of the PATp. The algorithm denoted by “Alg. 1” represents the fully implemented estimation algorithm utilizing both PATs and HR.

It can be observed that among all the PAT-based methods, the PATs-based algorithm may be the best choice in terms of reducing (or possibly minimizing) the standard deviations. Among all the methods, the “Alg. 1” obtains the best performance with respect to the standard deviations since it combines both PATs and HR. The AAMI requirements for the BP estimation indicate that the mean of the estimation error has to be lower than 5 mmHg in absolute value, and that the standard deviation of the error has to be below 8 mmHg, for both SBP and DBP. This implies that the mean-square error has to be less than 89 mmHg. It can be observed from FIG. 9 that the only algorithm that meets the AAMI requirements on average is the “Alg. 1” algorithm. All other algorithms fail in satisfying the SBP standard deviation condition of being below 8 mmHg, while the “Alg. 1” algorithm achieves the standard deviation of 7.77 mmHg.

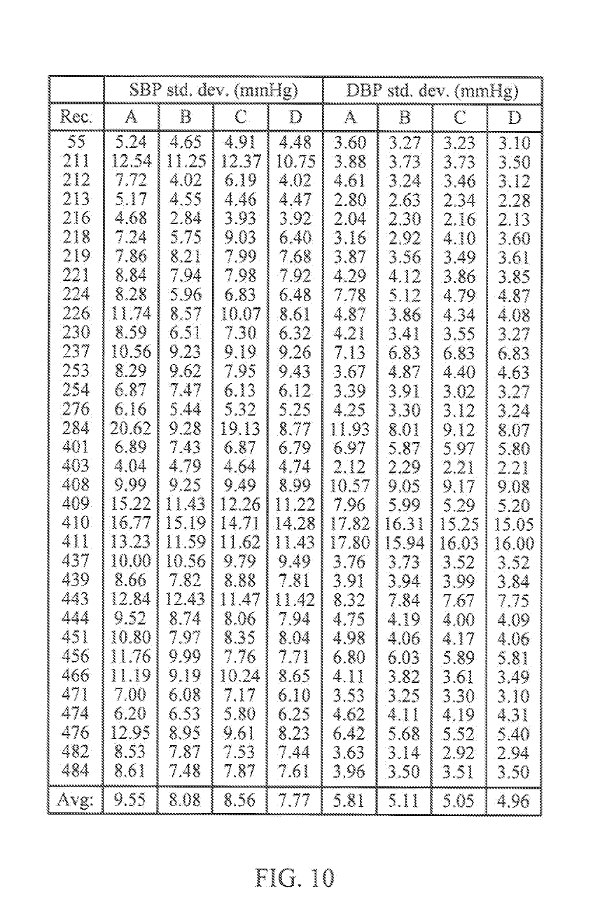

FIG. 10 illustrates the standard deviation of the SBP error for each of the 34 records from the MIMIC database, and for four estimation algorithms A, B, C, and D corresponding to the “No Est.”, “PATs only”, “HR only” and “Alg. 1”, respectively. It can be observed that the “Alg. 1” algorithm provides the best performance across all methods for most patients, though for some patients other methods may give better performance. When the “PATs only” method performs better than the “HR only” method (e.g., for the patent record 212), the “Alg. 1” may approach or improve the standard deviation value of the “PATs only” algorithm, and vice versa, when the “HR only” method is better than the “PATs only” method (e.g., the patient record 474), the “Alg. 1” solution still does better.

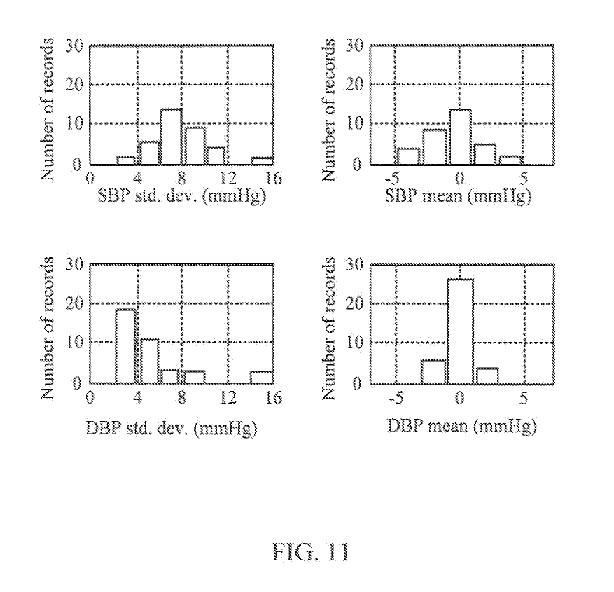

Distributions of the mean and standard-deviation of error for different patient records are illustrated in FIG. 11. It can be observed that for both SBP and DBP each record has an error with mean between −5 mmHg and 5 mmHg Regarding standard deviations of the DBP, most records are below 8 mmHg and four records do not meet the AAMI requirement. For the SBP, 14 records do not meet the AAMI requirement.

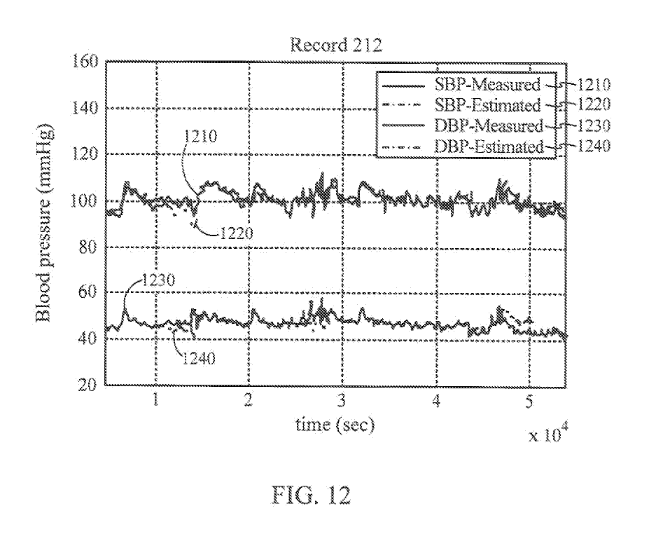

FIG. 12 illustrates the measured and estimated blood pressure waveforms for a portion of the patient record 212. It can be observed that the signals are in close agreement in this case, and the estimated waveforms (i.e., plots 1220 and 1240) follow closely the trends of the measured pressure (i.e., plots 1210 and 1230).

The calibration period T_{cal} may affect the estimation performance. The quantity T_{cal} denotes the time interval between consecutive re-calibration instances. The value of T_{cal} may depend on the specific application and the desired level of accuracy. More frequent calibrations may reduce the error while less frequent calibrations may make the system more amenable for everyday use.

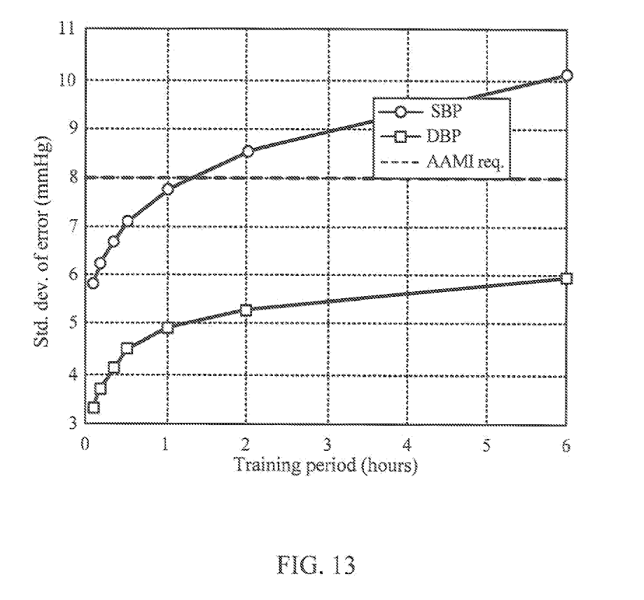

FIG. 13 illustrates the standard deviation of the estimation error for both the SBP and DBP as a function of T_{cal}. For small calibration periods, the true blood pressures are known more often and the error may be reduced. It can be observed from FIG. 13 that in order to meet the AAMI standard deviation requirement of 8 mmHg, T_{cal} may need to be approximately 1 hour and 20 min.

A skew may also affect performance of the proposed BP estimation algorithm. The skew refers to any time error produced when calculating the PAT. The skew may be especially important in Body Area Networks. In the BAN setup, it is possible to have a node in the chest taking ECG measurements, and a different node in the finger taking PPG measurements. Assuming that these two nodes send their measurements to a concentrator such as a PDA or a cell-phone, the synchronization between the measurements may become an important issue. Specifically, the Quality of Service (QoS) grants for measuring sensors may result in unknown jitter and latency in reception at the receiver, and may result in errors in estimating the PAT from ECG and PPG signals.

Estimation error can be analyzed when the skew is artificially introduced in the PAT. Two types of skew can be considered: a random skew and a constant skew. The random skew represents random variations in the synchronization of the two signals caused by: a clock jitter, a processing delay, a transmission delay, a transmission jitter, etc. The added random skew has zero-mean and Gaussian distribution with variance σ_{τ}².

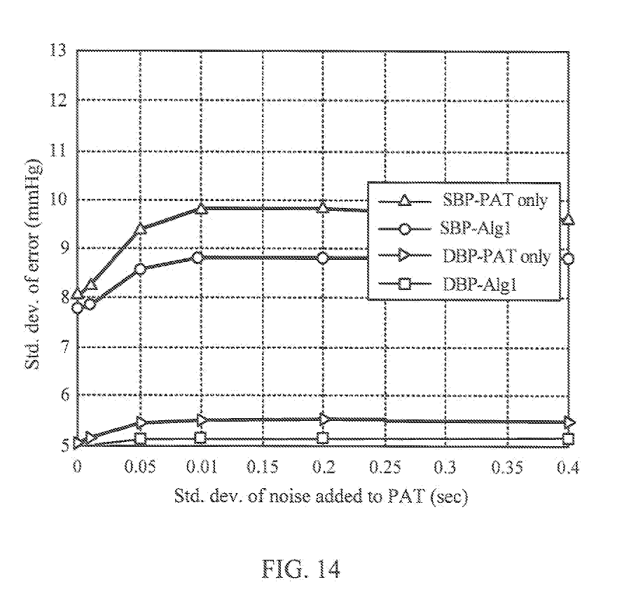

FIG. 14 illustrates the standard deviation of the estimation error as a function of the variance of the added skew. It can be observed that the skew may degrade the performance of the proposed algorithm up to a point where the degradation saturates. This represents the point where the PAT is too noisy and the algorithm starts relying on HR only to perform the blood pressure estimation. Since the HR can be measured from one signal only (either ECG or PPG signal), there may be no additional skew introduced in this signal due to the communication system.

The effect of adding a constant skew to the PAT is also studied in the present disclosure. In this scenario, the initial calibration of the system using measurements without skew can be performed. Subsequently, a constant skew to the PAT is introduced and the proposed estimation algorithm is applied. Two quantities are of interest. First, it is important to find out how the skew affects the estimation of blood pressure before any recalibration is performed. Second, it is important to know if the proposed re-calibration can mitigate the skew.

After the system has been calibrated for each patient, the estimation algorithm can be applied over one segment of duration T_{cal} without re-calibration. Then, the standard deviation and mean of the error on this first segment can be recorded, and this can be referred to as “segment 1”. Subsequently, the proposed re-calibration algorithm can be allowed to be applied on the signal, and the standard deviation and mean of the error can be recorded for all subsequent segments in the record excluding the segment 1.

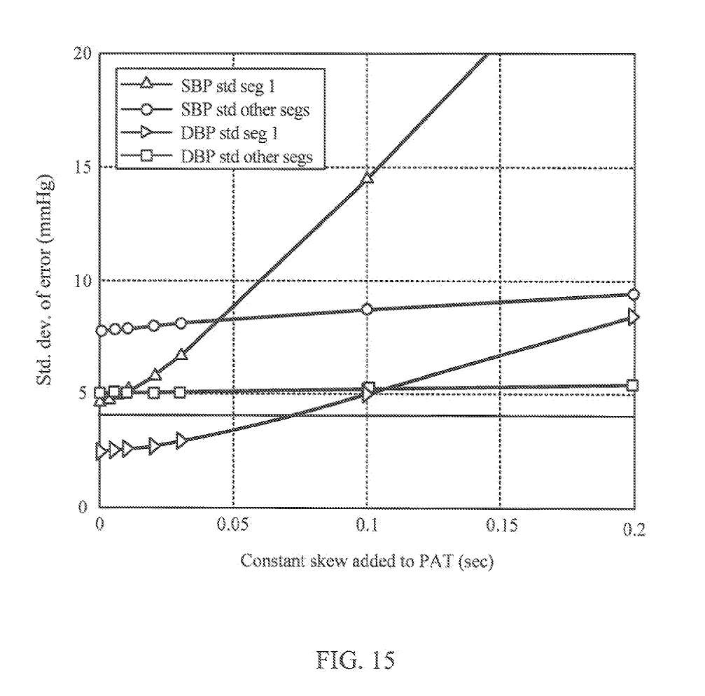

FIG. 15 illustrates effect of the skew on the standard deviation of the error for SBP and DBP for the segment 1 and remaining segments as a function of the amplitude of the added skew. It is important to note that both SBP and DBP estimates degrade considerably for the segment 1 as the skew increases. Skews of about 40 ms would be tolerable in this respect. Nonetheless, the standard deviation for the remaining segments does not change much as the amplitude of the skew is increased. This may indicate that the re-calibration algorithm is performing accurately, and the algorithm may be able to correct the skew after a few re-calibration instances. The exemplary simulations show that one re-calibration may be sufficient.

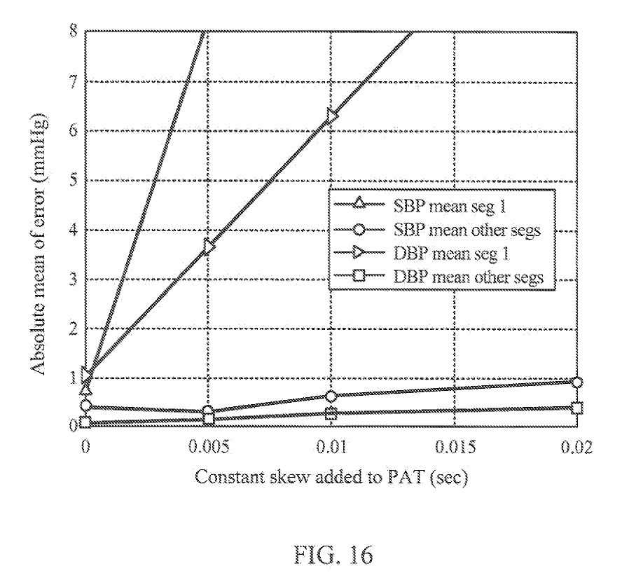

FIG. 16 illustrates effect of the skew on the absolute mean of the error for estimating SBP and DBP for the segment 1 and remaining segments as a function of the amplitude of the added skew. Again, the SBP and DBP estimates may degrade considerably for the segment 1 as the skew increases, and the degradation is more significant than that of the standard deviation. Skews of about 2-3 ms would be tolerable in this case. However, as was the case before for the standard deviation, the absolute mean for the remaining segments may remain within the required 5 mmHg, indicating that the skew is being corrected by the re-calibration algorithm.

It has been validated that the PAT provides an advantage to estimate SBP and DBP by using the MIMIC database recordings. Among all the parameters used for estimation including PATp, PATs, PATf, heart rate, mean of PPG, etc., it is found in the present disclosure that the parameter that correlates the best with SBP and DBP is the PATs (i.e., the PAT measured to the maximum slope point in the waveform). It is also found that the “HR only” estimation method perform for some patients even better than any PAT based methods. Thus, the estimation method is proposed in the present disclosure based on measuring PATs and HR achieving standard deviation of 7.77 mmHg for SBP estimation, assuming calibration every 1 hour. This is within the AAMI error requirement of 8 mmHg.

Regarding the estimation models, it is found in the present disclosure that a simple linear relation given by equation (7) may provide the best results across all patients. Even when relations such as the one given by equation (5) are better for some patients, they are not very robust to noisy measurements and may therefore produce high estimation errors for other patients.

It is shown in the present disclosure that the calibration time required to achieve BP estimation results well within the AAMI requirements may be approximately equal to 1 hour and 20 minutes. When the calibration is performed, for example, every 6 hours, then the standard deviation of the estimation error may raise above 10 mmHg.

It is also shown in the present disclosure that the random skew between ECG and PAT may have the strong influence on estimation methods based on PAT only, but it may have less impact on the proposed estimation method which also utilized the instantaneous heart rate. This is because the heart rate may be obtained from the ECG only, and therefore it may be independent of the skew. Moreover, the effect of adding the constant skew after the system has been trained is studied in the present disclosure. It is shown that significant degradation can be expected for skews greater than 2-3 ms, though this degradation can be corrected on the first re-calibration instance. Thus, a system that allows frequent recalibrations can be robust to constant skews.

The use of PAT and instantaneous HR to estimate SBP and DBP is proposed in the present disclosure. The proposed algorithm may estimate the parameters through an initial training. The model parameters may be re-calibrated at constant intervals using the recursive least square (RLS) approach combined with smooth bias fixing. The algorithm can be applied on the MIMIC database, and the results can be compared with estimation methods that use PAT only or HR only. It is found that the proposed algorithm meets the AAMI requirements on average, and outperforms other methods. It is also shown in the present disclosure that the proposed estimation algorithm can be robust to unknown skew between the ECG and PPG signals.

The various operations of methods described above may be performed by any suitable means capable of performing the corresponding functions. The means may include various hardware and/or software component(s) and/or module(s), including, but not limited to a circuit, an application specific integrate circuit (ASIC), or processor. Generally, where there are operations illustrated in Figures, those operations may have corresponding counterpart means-plus-function components with similar numbering. For example, blocks 710-730 and 810-870, illustrated in FIGS. 7 and 8 correspond to circuit blocks 710A-730A and 810A-870A illustrated in FIGS. 7A and 8A.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like. Also, “determining” may include measuring, estimating and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various operations of methods described above may be performed by any suitable means capable of performing the operations, such as various hardware and/or software component(s), circuits, and/or module(s). Generally, any operations illustrated in the Figures may be performed by corresponding functional means capable of performing the operations.

The various illustrative logical blocks, modules and circuits described in connection with the present disclosure may be implemented or performed with a general purpose processor, a digital signal processor (DSP), an application specific integrated circuit (ASIC), a field programmable gate array signal (FPGA) or other programmable logic device (PLD), discrete gate or transistor logic, discrete hardware components or any combination thereof designed to perform the functions described herein. A general purpose processor may be a microprocessor, but in the alternative, the processor may be any commercially available processor, controller, microcontroller or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the present disclosure may be embodied directly in hardware, in a software module executed by a processor, or in a combination of the two. A software module may reside in any form of storage medium that is known in the art. Some examples of storage media that may be used include random access memory (RAM), read only memory (ROM), flash memory, EPROM memory, EEPROM memory, registers, a hard disk, a removable disk, a CD-ROM and so forth. A software module may comprise a single instruction, or many instructions, and may be distributed over several different code segments, among different programs, and across multiple storage media. A storage medium may be coupled to a processor such that the processor can read information from, and write information to, the storage medium. In the alternative, the storage medium may be integral to the processor.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware or any combination thereof. If implemented in software, the functions may be stored as one or more instructions on a computer-readable medium. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, include compact disc (CD), laser disc, optical disc, digital versatile disc (DVD), floppy disk, and Blu-ray® disc where disks usually reproduce data magnetically, while discs reproduce data optically with lasers.

Thus, certain aspects may comprise a computer program product for performing the operations presented herein. For example, such a computer program product may comprise a computer readable medium having instructions stored (and/or encoded) thereon, the instructions being executable by one or more processors to perform the operations described herein. For certain aspects, the computer program product may include packaging material.

Software or instructions may also be transmitted over a transmission medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of transmission medium.

Further, it should be appreciated that modules and/or other appropriate means for performing the methods and techniques described herein can be downloaded and/or otherwise obtained by an access terminal and/or access point as applicable. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that an access terminal and/or access point can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes and variations may be made in the arrangement, operation and details of the methods and apparatus described above without departing from the scope of the claims.

A wireless device in the present disclosure may include various components that perform functions based on signals that are transmitted by or received at the wireless device. A wireless device may also refer to a wearable wireless device. In some aspects the wearable wireless device may comprise a wireless headset or a wireless watch. For example, a wireless headset may include a transducer adapted to provide audio output based on data received via a receiver. A wireless watch may include a user interface adapted to provide an indication based on data received via a receiver. A wireless sensing device may include a sensor adapted to provide data to be transmitted via a transmitter.

A wireless device may communicate via one or more wireless communication links that are based on or otherwise support any suitable wireless communication technology. For example, in some aspects a wireless device may associate with a network. In some aspects the network may comprise a personal area network (e.g., supporting a wireless coverage area on the order of 30 meters) or a body area network (e.g., supporting a wireless coverage area on the order of 10 meters) implemented using ultra-wideband technology or some other suitable technology. In some aspects the network may comprise a local area network or a wide area network. A wireless device may support or otherwise use one or more of a variety of wireless communication technologies, protocols, or standards such as, for example, CDMA, TDMA, OFDM, OFDMA, WiMAX, and Wi-Fi. Similarly, a wireless device may support or otherwise use one or more of a variety of corresponding modulation or multiplexing schemes. A wireless device may thus include appropriate components (e.g., air interfaces) to establish and communicate via one or more wireless communication links using the above or other wireless communication technologies. For example, a device may comprise a wireless transceiver with associated transmitter and receiver components (e.g., transmitter 210 or 302 and receiver 212 or 304) that may include various components (e.g., signal generators and signal processors) that facilitate communication over a wireless medium.

The teachings herein may be incorporated into (e.g., implemented within or performed by) a variety of apparatuses (e.g., devices). For example, one or more aspects taught herein may be incorporated into a phone (e.g., a cellular phone), a personal data assistant (“PDA”) or so-called smartphone, an entertainment device (e.g., a portable media device, including music and video players), a headset (e.g., headphones, an earpiece, etc.), a microphone, a medical sensing device (e.g., a biometric sensor, a heart rate monitor, a pedometer, an EKG device, a smart bandage, etc.), a user I/O device (e.g., a watch, a remote control, a light switch, a keyboard, a mouse, etc.), an environment sensing device (e.g., a tire pressure monitor), a monitoring device that may receive data from the medical or environment sensing device (e.g., a desktop, a mobile computer, etc.), a point-of-care device, a hearing aid, a set-top box, or any other suitable device. The monitoring device may also have access to data from different sensing devices via connection with a network.

These devices may have different power and data requirements. In some aspects, the teachings herein may be adapted for use in low power applications (e.g., through the use of an impulse-based signaling scheme and low duty cycle modes) and may support a variety of data rates including relatively high data rates (e.g., through the use of high-bandwidth pulses).

In some aspects a wireless device may comprise an access device (e.g., an access point) for a communication system. Such an access device may provide, for example, connectivity to another network (e.g., a wide area network such as the Internet or a cellular network) via a wired or wireless communication link. Accordingly, the access device may enable another device (e.g., a wireless station) to access the other network or some other functionality. In addition, it should be appreciated that one or both of the devices may be portable or, in some cases, relatively non-portable. Also, it should be appreciated that a wireless device also may be capable of transmitting and/or receiving information in a non-wireless manner (e.g., via a wired connection) via an appropriate communication interface.

1. A method for estimating a blood pressure, comprising:
estimating a pulse arrival time (PAT);
estimating a heart rate (HR); and
estimating the blood pressure based on the estimated PAT and HR.

2. The method of claim 1, wherein estimating the blood pressure comprises:
estimating a set of parameters; and
applying the estimated set of parameters on the estimated PAT and HR.

3. The method of claim 2, wherein estimating the set of parameters comprises:
collecting at least one observation of blood pressure, PAT or HR; and
estimating the set of parameters based on the at least one observation of blood pressure, PAT or HR.

4. The method of claim 2, further comprising:
calibrating the estimated set of parameters by using an adaptive algorithm.

5. The method of claim 4, wherein calibrating the estimated set of parameters comprises:
collecting initial set of observations of at least one of PAT, HR or blood pressure;
computing initial estimates of the parameters using the collected initial set of observations of at least one of PAT, HR or blood pressure;
collecting another observation of at least one of PAT, HR or blood pressure; and
updating values of the parameters based on the computed initial estimates and on the collected another observation of at least one of PAT, HR or blood pressure according to the adaptive algorithm.

6. The method of claim 4, wherein the adaptive algorithm comprises the recursive least squares algorithm.

7. The method of claim 2, further comprising:
bounding each parameter of the estimated set of parameters using a first bound and a second bound, if the parameter is smaller than the first bound or the parameter is larger than the second bound.

8. The method of claim 2, further comprising:
correcting any bias in the estimated set of parameters.

9. The method of claim 1, wherein estimating the PAT comprises:
estimating a time period between a defined point in an electrocardiogram (ECG) signal and a defined point in a photoplethysmogram (PPG) signal.

10. The method of claim 1, wherein estimating the PAT comprises:
estimating at least one time period between at least one of: P peak, Q peak, R peak, S peak, or T peak of an electrocardiogram (ECG) signal and at least one point in a photoplethysmogram (PPG) signal.

11. The method of claim 1, wherein estimating the PAT comprises:
estimating a time period between a defined point in an acoustic signal and a defined point in a photoplethysmogram (PPG) signal.

12. The method of claim 1, wherein estimating the HR comprises:
estimating a time period between two consecutive peaks of an electrocardiogram (ECG) signal.

13. The method of claim 1, wherein estimating the HR comprises:
estimating a time period between two consecutive peaks of a photoplethysmogram (PPG) signal.

14. The method of claim 1, wherein estimating the HR comprises:
using an acoustic signal corresponding to heart sounds.

15. The method of claim 1, wherein the estimated blood pressure comprises at least one of a systolic blood pressure or a diastolic blood pressure.

16. An apparatus for estimating a blood pressure, comprising:
a first estimating circuit configured to estimate a pulse arrival time (PAT);
a second estimating circuit configured to estimate a heart rate (HR); and
a third estimating circuit configured to estimate the blood pressure based on the estimated PAT and HR.

17. The apparatus of claim 16, wherein the third estimating circuit configured to estimate the blood pressure comprises:
a fourth estimating circuit configured to estimate a set of parameters; and
a circuit configured to apply the estimated set of parameters on the estimated PAT and HR.

18. The apparatus of claim 17, wherein the fourth estimating circuit configured to estimate the set of parameters comprises:
a circuit configured to collect at least one observation of blood pressure, PAT or HR; and
an estimator configured to estimate the set of parameters based on the at least one observation of blood pressure, PAT or HR.

19. The apparatus of claim 17, further comprising:
a calibrating circuit configured to calibrate the estimated set of parameters by using an adaptive algorithm.

20. The apparatus of claim 19, wherein the calibrating circuit configured to calibrate the estimated set of parameters comprises:
a first circuit configured to collect initial set of observations of at least one of PAT, HR or blood pressure;
a computer configured to compute initial estimates of the parameters using the collected initial set of observations of at least one of PAT, HR or blood pressure;
a second circuit configured to collect another observation of at least one of PAT, HR or blood pressure; and
a processor configured to update values of the parameters based on the computed initial estimates and on the collected another observation of at least one of PAT, HR or blood pressure according to the adaptive algorithm.

21. The apparatus of claim 19, wherein the adaptive algorithm comprises the recursive least squares algorithm.

22. The apparatus of claim 17, further comprising:
a processing circuit configured to bound each parameter of the estimated set of parameters using a first bound and a second bound, if the parameter is smaller than the first bound or the parameter is larger than the second bound.

23. The apparatus of claim 17, further comprising:
a processing circuit configured to correct any bias in the estimated set of parameters.

24. The apparatus of claim 16, wherein the first estimating circuit is configured to estimate a time period between a defined point in an electrocardiogram (ECG) signal and a defined point in a photoplethysmogram (PPG) signal.

25. The apparatus of claim 16, wherein the first estimating circuit is configured to estimate at least one time period between at least one of: P peak, Q peak, R peak, S peak, or T peak of an electrocardiogram (ECG) signal and at least one point in a photoplethysmogram (PPG) signal.

26. The apparatus of claim 16, wherein the first estimating circuit is configured to estimate a time period between a defined point in an acoustic signal and a defined point in a photoplethysmogram (PPG) signal.

27. The apparatus of claim 16, wherein the second estimating circuit is configured to estimate a time period between two consecutive peaks of an electrocardiogram (ECG) signal.

28. The apparatus of claim 16, wherein the second estimating circuit is configured to estimate a time period between two consecutive peaks of a photoplethysmogram (PPG) signal.

29. The apparatus of claim 16, wherein the second estimating circuit configured to estimate the HR comprises:
a circuit configured to use an acoustic signal corresponding to heart sounds.

30. The apparatus of claim 16, wherein the estimated blood pressure comprises at least one of a systolic blood pressure or a diastolic blood pressure.

31. An apparatus for estimating a blood pressure, comprising:
means for estimating a pulse arrival time (PAT);
means for estimating a heart rate (HR); and
means for estimating the blood pressure based on the estimated PAT and HR.

32. The apparatus of claim 31, wherein the means for estimating the blood pressure comprises:
means for estimating a set of parameters; and
means for applying the estimated set of parameters on the estimated PAT and HR.

33. The apparatus of claim 32, wherein the means for estimating the set of parameters comprises:
means for collecting at least one observation of blood pressure, PAT or HR; and
means for estimating the set of parameters based on the at least one observation of blood pressure, PAT or HR.

34. The apparatus of claim 32, further comprising:
means for calibrating the estimated set of parameters by using an adaptive algorithm.

35. The apparatus of claim 34, wherein the means for calibrating the estimated set of parameters comprises:
means for collecting initial set of observations of at least one of PAT, HR or blood pressure;
means for computing initial estimates of the parameters using the collected initial set of observations of at least one of PAT, HR or blood pressure;
means for collecting another observation of at least one of PAT, HR or blood pressure; and
means for updating values of the parameters based on the computed initial estimates and on the collected another observation of at least one of PAT, HR or blood pressure according to the adaptive algorithm.

36. The apparatus of claim 34, wherein the adaptive algorithm comprises the recursive least squares algorithm.

37. The apparatus of claim 32, further comprising:
means for bounding each parameter of the estimated set of parameters using a first bound and a second bound, if the parameter is smaller than the first bound or the parameter is larger than the second bound.

38. The apparatus of claim 32, further comprising:
means for correcting any bias in the estimated set of parameters.

39. The apparatus of claim 31, wherein the means for estimating the PAT comprises:
means for estimating a time period between a defined point in an electrocardiogram (ECG) signal and a defined point in a photoplethysmogram (PPG) signal.

40. The apparatus of claim 31, wherein the means for estimating the PAT comprises:
means for estimating at least one time period between at least one of: P peak, Q peak, R peak, S peak, or T peak of an electrocardiogram (ECG) signal and at least one point in a photoplethysmogram (PPG) signal.

41. The apparatus of claim 31, wherein the means for estimating the PAT comprises:
means for estimating a time period between a defined point in an acoustic signal and a defined point in a photoplethysmogram (PPG) signal.

42. The apparatus of claim 31, wherein the means for estimating the HR comprises:
means for estimating a time period between two consecutive peaks of an electrocardiogram (ECG) signal.

43. The apparatus of claim 31, wherein the means for estimating the HR comprises:
means for estimating a time period between two consecutive peaks of a photoplethysmogram (PPG) signal.

44. The apparatus of claim 31, wherein the means for estimating the HR comprises:
means for using an acoustic signal corresponding to heart sounds.

45. The apparatus of claim 31, wherein the estimated blood pressure comprises at least one of a systolic blood pressure or a diastolic blood pressure.

46. A computer-program product for estimating a blood pressure, comprising a computer-readable medium comprising instructions executable to:
estimate a pulse arrival time (PAT);
estimate a heart rate (HR); and
estimate the blood pressure based on the estimated PAT and HR.

47. A medical sensing device, comprising:
a sensor configured to sense a plurality of signals;
a first estimating circuit configured to estimate a pulse arrival time (PAT) based on at least two of the sensed signals;
a second estimating circuit configured to estimate a heart rate (HR) based on at least one of the sensed signals;
a third estimating circuit configured to estimate a blood pressure based on the estimated PAT and HR; and
a user interface configured to provide an indication based on the estimated blood pressure.

48. A watch, comprising:
a first estimating circuit configured to estimate a pulse arrival time (PAT);
a second estimating circuit configured to estimate a heart rate (HR);
a third estimating circuit configured to estimate a blood pressure based on the estimated PAT and HR; and
a user interface configured to provide an indication based on the estimated blood pressure.

49. A monitoring device, comprising:
a connector;
a receiver configured to receive via the connector a plurality of signals;
a first estimating circuit configured to estimate a pulse arrival time (PAT) based on at least two of the received signals;
a second estimating circuit configured to estimate a heart rate (HR) based on at least one of the received signals;
a third estimating circuit configured to estimate a blood pressure based on the estimated PAT and HR; and
a user interface configured to provide an indication based on the estimated blood pressure.
